(19) United States
(12) Patent Application Publication
(10) Pub. No.: US 2010/0121314 A1
(43) Pub. Date: May 13, 2010

(54) REGULATED DRUG DELIVERY SYSTEM

(76) Inventor: Mario Iobbi, Agoura, CA (US)

Correspondence Address: BERLINER & ASSOCIATES, 555 WEST FIFTH STREET, 31ST FLOOR, LOS ANGELES, CA, 90013, US

(21) Appl. No.: 12/444,836

(22) PCT Filed: Oct. 8, 2007
(86) PCT No.: PCT/EP07/60630

§ 371 Date: Jan. 20, 2010

(30) Foreign Application Priority Data
Oct. 12, 2006 (GB) 0620334.3
May 18, 2007 (GB) 0709545.8

(51) Int. Cl. A61M 5/00

(52) U.S. Cl. 604/890.1

(57) ABSTRACT

A drug delivery device for regulating delivery of a drug to a patient (105) provides a controlled rate of delivery which accounts for changes in health or required dose and, in systems with inherent lag, enables a rapid and accurate response whilst maintaining system stability. The device comprises a drug delivery or dose regulator (103); a sensor (107) for measuring a biochemical or physiological property associated with the drug or the condition to be treated; and a controller (109) configured to control the rate of delivery or dose of drug via the regulator in response to the difference in a measured biochemical or physiological property with respect to a target. In order to maintain the system stability, the controller comprises an anti-wind up component (225) for minimizing wind up effects, and/or a filter sub-component (231) to ensure that the controller does not generate output signals to control the regulator in response to noise or erroneous signals.

### FIELD OF THE INVENTION

The invention relates to a system for controllably administering a drug to a patient using closed loop feedback response driven regulation. The invention further relates to a device for the controlled delivery of a drug to a patient, a controller for use in such a device or system and a method of treatment of a human condition by controllable delivery of a drug using a closed loop feedback. More particularly, the invention relates to control mechanism for sluggish closed loop systems. The invention finds particular application in the delivery or administration of oxygen (or supplemental oxygen) to a subject, especially a patient in need of supplemental oxygen.

### BACKGROUND OF THE INVENTION

In the treatment or prophylaxis of many conditions, it is necessary to administer repeat doses of, or to intermittently or continuously deliver, a drug to maintain the concentration of a drug, biochemical component or a physiological or biological marker, in order to maintain a physiological or biological effect or to maintain the health of the patient or prophylactic effect being sought. However, in most situations the rate of infusion or delivery of the drug is pre-determined either by the properties of the drug delivery device or formulation or by a pre-set rate of infusion or delivery as prescribed by a medical practitioner.

There are circumstances, however, in which such pre-determined rates of drug delivery are inappropriate, which can lead to unnecessary patient discomfort, worsening of symptoms, complications and increased need for medical attention, especially emergency medical attention.

For example, some drug delivery regimes are variable from patient to patient, depending upon the rate of internal transport of a drug (e.g. a macromolecule), the rate of metabolism of a quickly absorbed active component, the other medications that the patient might be receiving, the level of activity the patient may be undertaking etc.

Variations can also occur depending upon the relative health of the patient and the activities they are engaged in (and relative level of activity). This is particularly the case, for example, in people receiving supplemental oxygen or on oxygen therapy, especially home oxygen therapy (typically considered to be supplemental oxygen provided on a continuous or intermittent basis in the home environment) or ambulatory oxygen therapy. Patients with Chronic Obstructive Pulmonary Disease (COPD), a condition which is characterized by progressive obstruction to airflow in the lungs typically from emphysema and/or chronic bronchitis, are often treated with supplemental oxygen or oxygen therapy. For these patients, treatment with supplemental oxygen to reverse hypoxaemia (low blood-oxygen level) can lead to reduced pulmonary artery pressure, alleviation of right heart failure, a strengthening of cardiac function and an increased tolerance to exercise, thereby providing an improved survival benefit. Long term oxygen therapy (LTOT) has been shown to increase survival among patients with COPD, although, even in patients on oxygen therapy, periods of hypoxemia can have adverse effects leading to right ventricular hypertrophy from increased pulmonary artery pressure and pulmonary vascular resistance, among other complications.

In practice, the oxygen therapy provided to patients is typically at a fixed dose of oxygen prescribed by a medical practitioner at the clinic and may be reviewed several times per year. Given the variation in health among patients, the fact that many patients suffer from their lungs' diminished ability for gas exchange performance to variable degrees and the variation in a patient's requirements, depending upon level of exercise or stress, the worsening of their condition or secondary respiratory conditions such as a cold, many patients find themselves in a hypoxic condition for at least a part of the day, feeling discomfort and/or their intended activities compromised.

Several potential solutions to these issues have been proposed, including several that involve closed loop control systems. Closed loop control systems are known in electronics in order to elicit a change or response to a measured parameter and often involve linear or PID (proportional, integral, differential) control algorithms (hereby referred to as active components). However, for some closed loop systems with inherent delays, such as drug delivery, where the measured parameter only responds after a significant delay from the controlled activity that influences it, a linear controller may be ineffective, unstable or even potentially dangerous and an effective and fast response cannot be delivered.

EP-B-1342482 relates to an implantable and controlled drug delivery system, having closed loop feedback control, which directly measures and responds to a detected biochemical parameter (related to the underlying medical condition), especially for the treatment of CNS disorders, in response to which a delivery device, such as an infusion pump, is triggered to deliver one or more drugs of an appropriate duration. The sensor in EP-B-1342282 may optionally detect the concentration of an infused drug or metabolite, pH, a molecule, gas or indicator thereof. In response to the measured biochemical parameter, the controller unit sends a signal to the pump assembly to deliver the recalculated flow of drug. The system is said to find particular utility for controlling delivery of large molecules with low transport rates and small molecules for drug protocols where predictive dosing may vary greatly, such as for the treatment of Parkinson's or other CNS disorders. There is no disclosure of how rapidly and accurately the closed loop feedback system can respond and how a rapid response and stability can be achieved.

WO 99/04841 is concerned with providing a sub-acute patient receiving supplementary oxygen with a dosage of oxygen for maintaining healthy blood-oxygen saturation levels whilst simultaneously conserving oxygen by providing a demand delivery system with a feedback control mechanism. The mechanism described is to continuously measure arterial blood oxygen saturation and to restrict delivery of supplemental oxygen to the patient if the blood oxygen level is above a predetermined level and to deliver oxygen if the level falls below a pre-determined minimum. The dose of oxygen provided may vary at least partially according to the disparity between the desired and measured blood oxygen levels. A PID control scheme is envisaged for determining the desirable dose of oxygen to be delivered in response to the patient's need. The dosage of oxygen provided is determined by the duration of supply of a fixed flow of oxygen through a valve having ON/OFF positions during patient inhalation. The feedback loop ensures that when a patient has a healthy blood oxygen level, unnecessary oxygen is not wasted and the demand system ensures oxygen is not wasted during patient exhalation. The system is capable of significantly improving oxygen conservation over a standard continuous flow system. There is no discussion as to how a rapid response can be achieved whilst maintaining system stability. Furthermore, the demand driven systems have been shown to have significant differences in efficacy depending upon the nature of the system and the type of bolus delivered (see, for example, Roberts et al, Thorax, 51(8), 831, 1996 & Fuhrman et al, Respiratory Medicine, 98(10), 938, 2004).

WO 00/18460 describes an oximetry device and method for delivering a controlled flow of supplementary oxygen to a patient receiving oxygen therapy according to a measured blood-oxygen saturation level. The described device comprises an oximeter for measuring blood oxygen saturation, which is communicated to a controller which compares the measured level to a pre-determined range and if above or below the target range instructs a valve located in an oxygen delivery conduit to decrease the flow rate in a stepwise manner with pre-determined sized steps (e.g. by 0.5 l/min). The device further comprises a memory for storing oxygen saturation and flow rate data and optionally an interface that is communicable with an external or remote data device, which may enable remote monitoring of a patient by a physician, including remote manipulation of the desired range limits (e.g. the oxygen prescription). This does not address the time lag issue nor provide rapid correction of oxygen saturation relative to a desired oxygen saturation since it enables the oxygen delivery only to be adjusted in a progressive stepwise manner. This can take a significant amount of time to change the valve from fully closed to fully open in response to blood-oxygen saturation readings. Accordingly, the issue of rapid response whilst maintaining a stable system is not addressed since the response provided is slow and incremental and not able to cope with large or sudden changes.

WO 2006/014399 is concerned with a method an apparatus for monitoring and conserving the supply of respiratory oxygen to a patient by using a dual sensor oxygen therapy device. The device utilizes a blood-oxygen saturation sensor from which the error from a pre-determined setpoint is calculated and a first responsive signal generated using a slow-acting oxygen saturation feedback controller and a pulse rate sensor from which the change in pulse and thereby theoretical activity of the patient is detected by a feed forward controller which generates a second responsive signal in anticipation of the change in patient oxygen need. The two responsive signals are combined and sent to an oxygen controller's variable frequency power source, whereupon the predicted amount of oxygen required is produced by the concentrator and delivered to the patient. There is not described any direct solution to the lag issue associated with prior saturation-driven feedback controllers and instead the use of pulse rate change is utilized as a predictive measure, although how the pulse rate is used to predict the change in oxygen supply provided is not described. It assumes a direct and predictive causal link between blood-oxygen saturation and heart rate. Furthermore, this scheme would be detrimental to a patient's blood oxygen saturation should the pulse rate be slowing whilst the patient is desaturating.

Whilst many prior art documents describe devices that utilize feedback control to increase or decrease the dosage of a drug to a patient in response to a measured parameter, many, especially for oxygen therapy, are more focused on conservation of oxygen and the energy used in producing oxygen. There is no adequate means for ensuring that the supply of a drug, especially oxygen, to a patient is controllably varied in a rapid and accurate manner in response to a measured biochemical, biological or physiological parameter affected by the drug being delivered.

### PROBLEM TO BE SOLVED BY THE INVENTION

There is therefore a need for a drug delivery system that can provide accurate and responsive therapy according to a patient's changing requirements as determined by a biological or biochemical effect or event, whilst providing rapid response and maintaining stability of the system.

It is a further object to provide such a system effective despite potentially long feedback response lag or dead time.

It is an object of the present invention to provide a controller capable of providing an accurate and rapid response in dose or delivery rate to a patient to measured biochemical, biological or physiological parameters whilst maintaining system stability, particularly when the measured parameter is characterized by a delayed response or time-lagged with respect to the stimulus.

It is a further object of the present invention to provide a system for regulated administration of a drug utilizing such a controller and a method of treating a medical condition, in which a the system for regulated administration of a drug enables an improved treatment regime for a patient.

### SUMMARY OF THE INVENTION

In accordance with a first aspect of the invention, there is provided a drug delivery device for regulating delivery of a drug to a patient, the device comprising a regulator for controllably varying the rate of delivery or dose of a drug passing to the patient; at least one sensor for measuring a biochemical, biological or physiological property of the patient, said biochemical, biological or physiological property being associated with the drug to be delivered or with a condition to be treated by the drug to be delivered, and generating a feedback signal corresponding to said measured biological or physiological property; a controller, in communication with the at least one sensor and the regulator, configured to control the rate of delivery or dose of the drug provided by the regulator in response to one or more measured biochemical, biological or physiological properties provided by the at least one sensor with respect to a predetermined target, the controller comprising a comparator having a signal input configured to receive a feedback signal from at least one sensor corresponding to a value of a measured biological or physiological property and being capable of generating an error signal corresponding to the disparity between a target value and a feedback signal; an active control component for generating an active output signal in response to the error signal; and a manipulation component for manipulating the active output signal and generating a post-manipulation (or manipulated output) signal for communicating to the regulator as a final output signal, characterized in that the controller further comprises an anti-wind up component and/or the manipulation component comprises a filter sub-component for conditioning the active output signal; whereby the final output signal provides a controlled rate of delivery or dosage administered to a patient in response to the measured biochemical, biological or physiological property.

In a second aspect of the invention, there is provided a delivery system for regulated administration of a drug to a patient, the system comprising a source or reservoir of a drug; a drug delivery means for the passage of a drug from the source or reservoir to a patient; and a drug delivery device as defined above, which is configured such that the regulator is for controllably varying the rate of delivery or dose of drug passing to the patient via the drug delivery means from the source or reservoir.

In a third aspect of the invention, there is provided a feedback controller comprising a comparator having a signal input configured to receive an input signal corresponding to a value of a parameter to be influenced and being capable of generating an error signal corresponding to the disparity between a target value and an input signal; a communication means for communicating a final output signal receivable by a component capable of regulating an activity affecting the parameter to be influenced; an active control component having a proportionally responsive sub-component for providing a proportional response to an error signal received from the comparator and an integrally responsive sub-component for providing an integral response to the error signal, which active control component is capable of generating an active output signal corresponding to a combination of the responses of its sub-components; an error signal tuner, which comprises of a proportional gain to generate an amplified proportional signal and an integral gain to generate a pre-integration signal; a manipulation component for manipulating the active output signal, the pre-manipulation signal and generating a post-manipulation signal for communicating via the communication means as a final output signal; and an anti-wind up component comprising a signal disparity component which generates an anti-wind up signal from the difference between a pre-manipulation signal and a post-manipulation signal, and optionally a signal gain for amplifying the anti-wind up signal, which is combined with the pre-integration signal of the integral component of the active component; whereby the final output signal is such as to provide a rapid and accurate response to a time-delayed or time-lagged measured parameter whilst maintaining system stability.

In a fourth aspect of the invention, there is provided a method of administering a drug to a patient comprising the steps of providing a source or reservoir of a drug; providing and fitting to a patient a delivery means for passage of the drug from the reservoir or source to the patient; providing a device as defined above configured to control the passage of the drug from the reservoir or source to the patient via the drug delivery means and fitting the at least one sensor of the device to the patient and configuring said sensor to measure a biochemical, biological or physiological parameter to be influenced by the administration of the drug; administering a dose or delivery rate of the drug to the patient as regulated by the device; and causing the controller of the device to control administration of drug to the patient in response to signals generated by the sensor, whereby the rate of delivery or dosage administered to the patient is such as to provide a rapid and accurate influence on the measured biochemical, biological or physiological property as desired.

In a fifth aspect of the invention, there is provided a method of therapeutic, prophylactic or diagnostic treatment of a human or animal body comprising administering to a patient a drug according to the method defined above.

In a sixth aspect of the invention, there is provided a method of treating a patient having COPD or otherwise in need of continuous or intermittent oxygen therapy, the method comprising administering to a patient a supply of oxygen by the method defined above, wherein the drug is oxygen and the source of the drug is an oxygen cylinder or an oxygen concentrator.

### ADVANTAGES OF THE INVENTION

The present invention enables a rapid and accurate closed loop feedback response to the effect of an amount of an administered drug on a measurable parameter in the body, whilst maintaining system stability. By utilizing the system, device, controller or method of the invention, a patient receiving a prophylactic or therapeutic administration of a drug, the amount of which has a proportional or critical effect on the body, may be delivered the effective amount of a drug on a feedback response basis rather than the predicted amount of the drug. The invention thus enables effective drug administration over a prolonged period which accounts for changes (deterioration or improvement) in the health of the patient, changes in the required dosage and state of activity of the patient. The invention has consequential benefits for health care professionals in terms of saved time and patient satisfaction and cost benefits for health care providers.

The invention is particularly beneficial for use in the treatment or prevention of conditions where the measurable effect is time delayed with respect to the provision of the drug causing the effect, since it enables as rapid and accurate a response to the effect that the time delay will allow, without causing the system to become unstable or to issue erroneous drug delivery instructions.

The invention finds particular advantages in the delivery of oxygen to patients receiving supplementary oxygen in a home oxygen therapy environment or in ambulatory, including emergency, environments.

### BRIEF DESCRIPTION OF THE DRAWINGS

FIG. 1 is a diagram illustrating an overview of a delivery system according to the present invention;

FIG. 2 is a representation of an example of a controller according to the present invention;

FIG. 3A is an illustration of a response by a measurable parameter (solid line) to a step change in an activity influencing that parameter (dashed line) in a system characterized with a time-lagged or time delayed response.

FIG. 3B illustrates a variation in a patient's arterial blood oxygen saturation with time when receiving oxygen via a feedback control system not able to cope with the time delayed response;

FIG. 3C illustrates a variation in oxygen flow via a feedback control system not able to cope with the time delayed response, which is delivering oxygen to the patient referred to under FIG. 3B.

FIG. 4A shows a graph of oxygen saturation over time for a patient receiving feedback controlled oxygen;

FIG. 4B shows a graph of oxygen flow over time delivered to the patient referred to under FIG. 4A;

FIG. 4C shows a graph of oxygen saturation pressure over time for a patient receiving feedback controlled oxygen with a device according to the present invention;

FIG. 4D shows a graph of oxygen flow over time delivered to the patient referred to under FIG. 4C with a device according to the present invention;

FIG. 5A shows a graphical representation of a variable error signal over time;

FIG. 5B shows a graphical representation of an output signal corresponding to the error signal of FIG. 5A having been manipulated by manipulation components; and

FIG. 5C is a bar chart illustrating the percentage of time a patient spends with detrimentally low oxygen according to three oxygen delivery regimes.

### DETAILED DESCRIPTION OF THE INVENTION

The system, device and methods of the present invention may be used in the provision of any drug to be administered to a patient in a sustained manner (i.e. in a continuous or intermittent manner over a sustained period) which causes a measurable effect in the body. It is particularly suited to applications where the concentration or quantity or volume of the drug is critical, or even proportional, to the benefits elicited by the drug. The present invention finds particular application where the measurable effect or parameter related to the drug being administered is time delayed or time-lagged with respect to the causal drug administration.

The controller of the present invention may be particularly beneficial when used for controlling any regulating activity that has a measurable effect that is time-lagged or characterized by a time delayed response. The controller, having an active component susceptible to integrator wind up in systems with a time lag or time-delayed response and/or where limits are placed upon the activity being regulated, is provided with an anti-wind up component for minimizing wind up effects and for maintaining system stability. Such a controller may preferably be used in the device of the present invention for regulating the delivery of a drug to a patient or for similar, non-medical devices and systems relating to the delivery of a substance affecting the body.

The system and device of the present invention may find particular utility in certain patients in whom the condition is chronic, the treatment is ongoing or regular and the necessary dose can change with time. For example, the invention may be useful for the treatment of diabetes by regulating the administration of insulin via a suitable device such as a pump injection device in response to a measure in blood-sugar levels with an appropriate diagnostic or sensor. As another example, the invention may be useful in the treatment of COPD by regulating the administration of oxygen to a patient via a valve in response to blood oxygen saturation measurements with a pulse oximeter as a sensor.

The device according to the present invention has as key components a regulator, a sensor and a controller as defined above. The system of the invention further comprises a drug reservoir or source and a drug delivery means.

The controller, which is capable of communicating with a regulator and receiving signals from one or more sensors, comprises of a comparator for generating an error signal from a received feedback signal (or input signal, received from a sensor, typically) with respect to a pre-determined target (or setpoint), a signal output (or communication means) for transmitting a final output signal, an active control component for providing an active response to the error signal and generating an active output signal and a manipulation component for manipulating the active output signal to generate a manipulated output signal for transmitting as the final output signal. The controller further comprises a filter sub-component of the manipulating component for conditioning the signal and/or, where the active control component is susceptible to wind up, an anti-wind up component which generates a signal fed back into the active controller which signal accounts for manipulations to the signal by one or more sub-components of the manipulation component. For example, the signal generated by the anti-wind up component may relate to the difference between a pre-manipulation signal and a final output signal. Unless any additional components are present which will modify the post-manipulation signal, then the final output signal is the post-manipulation signal.

The manipulation component may comprise of any number of sub-components, arranged in series or in parallel, which affect the active output signal to ultimately generate a final output signal. Each sub-component generates a respective manipulated signal (or post-manipulation signal) from the signal it receives, the manipulated signal designed to improve the final output signal in some way, directed toward improved response, performance, effect or signal handling.

Sub-components of the manipulation component may include, for example, a limit (or saturation) sub-component, one or more filter sub-component and/or a phase compensator. Each sub-component manipulates its pre-manipulation signal to generate its post-manipulation (or manipulated) signal.

A limit (or saturation) sub-component, which generates a post-limit (or post-saturation) signal from a pre-limit (or pre-saturation) signal, may be included to ensure that the output signal falls within certain minimum and maximum limits that may be defined. The limits might relate to the safe dosage of the drug being administered, to a range of doses prescribed by a clinician or to physical limitation of some aspect of the delivery system, such as the regulator. The limit (or saturation) sub-component is preferably included within the anti-wind up component. Whilst the effect on the dose of drug being delivered might not be directly affected by the limit (or saturation) sub-component if it is set to reflect the physical limitations of, for example, the regulator, including it within the anti-wind up component enables the anti-wind up signal to account for physical limitations as well as pure signal manipulations.

A filter sub-component, which generates a post-filtration signal from a pre-filtration signal (respective post- and pre-manipulation signals), may be included. The filter, which is intended to condition the signal for transmitting to the regulator, may include but is not restricted to a low pass filter (which filters out or attenuates signals above a specified frequency), a band pass filter (which filters out or attenuates signals below a specified minimum frequency and above a specified maximum frequency) and a high pass filter (which filters out or attenuates signal below a specified frequency). Preferably for applications where there is a time lag or time delay associated with the biochemical, biological or physiological effect being measured with respect to the action of the controller and the dose of drug delivered via the regulator, the filter is a low pass filter which disregards signals above a cut-off frequency, which is preferably a frequency which ensures that most received signals that are at a higher frequency than the associated time delay or time lag are disregarded. For example, in the delivery of supplemental oxygen for oxygen therapy, the delay or lag in the affected oxygen saturation as measured by a pulse oximeter to a change in dose administered may be X seconds. In this situation, the low pass filter cut-off frequency may be set such that signals received from the sensor having a frequency near or higher (quicker) than 1/(X seconds) are disregarded to ensure that the controller does not generate output signals in response to noise or spurious or erroneous signals, since higher frequency signals may be assumed not to have resulted from a change in dose of oxygen administered. Responding to such high frequency signals may cause potentially harmful controller instability, so the adoption of an appropriately configured low-pass filter may prevent such instability.

The filter sub-component may be of any suitable form. Preferably, the preferred low-pass filter for use in the present invention is a Butterworth filter and more preferably a first order Butterworth filter. This has the benefit of providing a fast response time.

A filter can improve the controller performance and stability over the allowable range of measured feedback response. A weighted average filter can suppress the effect of sporadic artifact measurement.

A phase compensator may optionally be included within the manipulation component. A phase compensator is useful for improving response time of the controller by compensating for phase lag generated by other components in the controller. In particular, the phase compensator can compensate for lag time generated by the filter. The phase compensator can be located within or outside the anti-wind up component. If the manipulation component comprises both a phase compensator and a filter, the two sub-components may be positioned in any order relative to each other, but preferably the phase compensator is located after the filter, particularly when the sensor feedback signal does not have high precision.

An anti-wind up component, which comprises a disparity component and a gain (or amplifier), generates an anti-wind up signal from the disparity between a pre-manipulation signal and a post-manipulation signal, which are preferably the active output signal and final output signal respectively.

Sub-components of the manipulation component that act after the pre-manipulation signal used by the anti-wind up and before the post manipulation signal used by the anti-wind up are said to be within the anti-wind up component, by which it is meant that the manipulation of those sub-components is accounted for in the anti-wind up signal generated.

The anti-wind up component has a benefit if at least one sub-component of the manipulation component is inside the anti-wind up component. Preferably, a limit (or saturation) sub-component is within the anti-wind up component and more preferably a filter sub-component is also within the anti-wind up component. Still more preferably, a saturation sub-component, a filter sub-component and a phase compensator are within the anti-wind up component. Most preferably, the manipulation component is located entirely within the anti-wind up component, by which it is meant that the pre-manipulation signal and post-manipulation signal used by the disparity component are the active out put and the final output respectively.

Preferably, the system, controller and device of the present invention comprise an anti-wind up component. The anti-wind up component inhibits, compensates for or prevents wind-up within a controller susceptible to signal wind up (e.g. error signal wind up). By inhibiting or preventing error signal wind up, the system becomes more responsive and more rapid since a change in the parameter being measured, e.g. by the sensor, has to result in controller wind-down prior to responding unless an anti-wind up facility is included. The inclusion of an anti-wind up component enables the controller to cope better with limits (e.g. the limits placed upon the output by the limit (or saturation) sub-component), which may otherwise cause wind up (particularly to an integral sub-component) and/or time delays resulting from feedback response delays.

The anti-wind up signal may be fed into the active controller as an amplified signal or may be fed into the appropriate gain of the tuner to affect the sub-component of the active component responsible for potential wind up. Preferably, the anti-wind up component has an amplifier so that the gain on the anti-wind up signal can be independently controlled.

Active components are typically susceptible to wind up when there is a sub-component that tends to sum or multiply (or integrate) the error signal. Active components having an integrator sub-component, for example, are susceptible to wind up. In controllers having an integral sub-component in the active component, the anti-wind up signal is preferably amplified to generate an amplified anti-wind up signal and combined with the pre-integration signal. Optionally, the amplified anti-wind up signal can be combined with the pre-integration signal before or after the integral portion of the tuner, but preferably after.

As an alternative embodiment to an anti-wind up component, the integral component can be periodically reset at pre-determined or set intervals, e.g. every 10 cycles. However, it is preferred to have an anti-wind up component since the degree of wind up and the extent of integrator benefit can not be consistently predicted to be divided by a pre-determined reset period and an anti-wind up component provides better stability and more rapid response.

Preferably the controller adopted in the device and system of the present invention comprises a linear feedback controller (e.g. a linear feedback algorithm) and the active component comprises a proportionally responsive sub-component for providing a proportional response to the error signal and an integrally responsive sub-component for providing integral responses to the error signal, which active control component generates an active output corresponding a combination of the responses provided by the sub-components of the active component and wherein the controller further comprises an error signal tuner, which comprises of a proportional gain to generate an amplified proportional signal, and an integral gain to generate a pre-integration signal.

The purpose of the error signal tuner is to adjust the response of the controller in proportion to the sensitivity of the system or subject (e.g. patient) or to the dynamics of the particular system. The skilled person in the art would be able to tune the gain parameters for a particular system or a particular drug regime to optimize the desired response benefits.

Whilst the tuner comprises amplifiers or gains for each of the proportional, integral and differential sub-components, as appropriate, of the active control component, should no amplification or gain be required for any particular sub-component, the gain of one may be applied or amplification, optionally may not be present.

In one preferred embodiment, the active component of the controller has a proportional sub-component, a differential sub-component and an integral sub-component, which may be tuned as appropriate using the error signal tuner, which has a gain associated with each of the sub-components of the active component.

The comparator, which generates an error signal corresponding to a difference between a received signal (e.g. a signal received from a sensor measuring a biological, biochemical or physiological property) and a target value, may have a hard wired or pre-programmed target value for the particular application of the controller. The comparator may have a single target or target value, multiple target values or may represent a target range of values. For example, the comparator may have target (or set-point) values representing a lower set-point and an upper set-point, whereby an error signal is only generated if the received signal is below the lower set-point or above the higher set-point. Alternatively and preferably for most applications, the target value is single desired value that the system seeks to achieve.

Preferably, the comparator has a target or set-point input means (e.g. a user interface) whereby the target can be changed for a particular application or in a change of circumstances (e.g. a change of health of a patient, a change of prescription of a drug to a patient, use of the device with another patient with different needs, etc). The target input means may be a simple dial, wheel, slide button or microprocessor interface, such as a keyboard or mouse and screen, e.g. a surface active screen.

The comparator has a signal input configured to receive an input signal corresponding to a measured value. The input signal may typically be a feedback signal generated by the at least one sensor.

The controller of the present invention may be provided by a microprocessor or as a hard-wired or analogue circuit, but preferably is provided by a microprocessor. In either case, a system clock will be accommodated, as appropriate, into the controller (and provided within the microprocessor in the preferred alternative).

The controller according to the present invention may be adapted for use in the device or system of the invention and for the drug delivery applications described or may be adapted for use in other medical applications, or other non-medical oxygen delivery applications. This may involve creating a control device having a regulator being controlled by the controller in response to signals received from one or more sensors. The use of such a device may optionally be applied in the same way on the device and system discussed above. Alternative applications for a controller according to the invention may be, for example, as part of an oxygen or air supply for use at altitude (e.g. for a pilot, such as a fighter pilot, altitude work or altitudes sport, such as climbing, or altitude training). Alternatively, the controller may be applied to sub-aqua applications where delivery of a gas or gas mixture helps ensure a user maintains healthy physiological function. In such applications, the preferred features and operations may be as for the other embodiments of the invention except that the system will typically be for provision of air or oxygen supply to a person who may not be a patient.

The regulator of the device of the present invention may be any means for controllably varying the delivery of a drug (or for non-medical applications, any means for controllably varying the extent of an activity having influence on a measurable parameter). For example, the regulator may be a valve, a titration apparatus, a drug infusion device, a pressure generating device upon a reservoir with a fixed aperture, etc. In a preferred embodiment, the regulator comprises a valve. The valve may be any suitable valve having a controllably variable aperture, which may be on/off adjustable, stepwise adjustable, or continually adjustable, preferably stepwise or continually adjustable and most preferably a continually adjustable valve such as a proportional solenoid valve.

Whilst, typically for closed loop feedback systems, the regulator's response does not need to be separately monitored, for time-lagged situations such as with supplemental oxygen therapy, it is preferable to separately monitor the performance and behaviour of the regulator or valve, since otherwise there could be a significant delay before the effects of an erroneous valve aperture are registered. By monitoring the behaviour and performance of the regulator, especially a valve, separately from the closed loop feedback controller, any harmful physiological effects from such an error can be minimized. Accordingly it is preferred that there is monitor of the dose of drug being delivered via the regulator, which in the case of a valve may be a flow meter. The monitor is preferably associated with the valve in a separate closed loop feedback control system, or alternatively provides an additional signal to the system controller for which a separate feedback loop can be created.

The sensor may be any means of measuring a parameter that is influenced by or associated with the activity controllably varied by the regulator (e.g. drug delivery) and of generating a signal corresponding to the measured parameter. The measurable parameter may be directly or indirectly influenced by the regulated activity, e.g. drug delivery/administration. For example, the parameter may be a biological, biochemical or physiological parameter. In the case of delivery of a drug, the parameter may be the concentration of the drug in the blood, the concentration of a metabolite, the concentration of a drug or subsequent signal at a particular point in the body, the presence of a component of a biochemical pathway affected by the drug (e.g. a signaling pathway), the change in blood pressure, heart rate or arterial blood oxygen saturation with, for example, oxygen (or carbon dioxide). Accordingly, the sensor may be, for example, a pulse oximeter, heart rate monitor or diagnostic device.

As mentioned above, the device and system according to the invention find particular utility when the biological, biochemical or physiological property being measured by the at least one sensor is time-lagged or time delayed with respect to the delivery of drug with which it is associated.

The system of the present invention, as mentioned above, comprises the described device in conjunction with a drug reservoir or source and a means for delivery of the drug.

The drug delivery means may be any suitable means for transporting the drug from the reservoir or source to the patient and which rate or dose of which transfer may be regulated by the regulator. For example, the drug delivery means may be a membrane, a catheter such as a multi-aperture catheter for implantation, an infusion device, an intravenous drip, a titration device or, for delivery of a gas, tubing with a mask for gas delivery or a nasal cannula.

The drug reservoir or source may be any means for storing, preparing or manufacturing the drug in an amount for use with the system. For example, the drug reservoir may be a vile of a drug or, where the drug is a medical gas, such as oxygen, a cylinder. The source may be, for example, an oxygen concentrator, where the drug to be administered is oxygen.

In a preferred embodiment of the invention the drug to be administered to a patient is a medical gas. The medical gas may be, for example, nitrogen oxide (NO) or oxygen. Preferably, the drug is oxygen for supplemental oxygen therapy.

The oxygen may also administered to the patient, for example, via a facial mask or nasal cannula.

By oxygen, for use as a drug in the system of the present invention, it is meant that the gas supplied has an enhanced level of oxygen as compared to the surrounding environment or atmospheric air. Preferably, the oxygen supply comprises at least 50% oxygen, more preferably at least 70% oxygen, still more preferably at least 80% oxygen and optionally 90% or 95% oxygen or more (e.g. pure oxygen).

By supplemental oxygen therapy, it is meant intermittent or continuous administration of enhanced oxygen-containing gas in the home, hospital or ambulatory environment. By supplemental oxygen therapy it is meant to include the provision of oxygen to patients with reduced lung function or suffering respiratory disorders in a variety of setting but not artificial ventilation of a patient using an artificial ventilator or iron lung.

The benefit of supplemental oxygen therapy is to prevent or reduce periods of hypoxaemia, or dangerously low blood oxygen saturation levels, in patients prone to hypoxaemia or suffering a condition having reduced lung function, which may otherwise cause pulmonary artery pressure, right heart failure, weakened cardiac function and reduced exercise tolerance.

By using a system according to the preferred embodiment of the present invention, oxygen administration can be provided according to the patient's needs as determined by a sensor measuring, for example, the level of oxygen saturation of the blood (e.g. using a pulse oximeter), more quickly and accurately than known methods. In particular, a system having a low pass filter to remove spurious signals and signals of higher frequency than the inherent lag in the measurement of oxygen saturation of the blood and an anti-wind up circuit incorporating such a filter, an overactive response is prevented and accurate and responsive administration of oxygen is provided whilst stability of the system maintained.

At present in the clinic, oxygen is prescribed at a fixed flow based upon, for example, a twenty minute titration in the doctor's surgery, which may include some exercise, e.g. waking on a treadmill. Based on this test, a fixed flow of oxygen is typically prescribed. For treating resting hypoxia 2 litres per minute might be prescribed for a provision of supplementary oxygen therapy, whilst an extra provision of 1 litre/minute above the resting flow rate is common for occurrences during exercise or sleep.

The oxygen therapy prescribed in this manner can often be inadequate for the patient as their need changes over time, according to their activity and according to their health, stress or excitement. It has been shown in a study by Morrison et al (“The adequacy of oxygenation in patients with hypoxic chronic obstructive pulmonary disease treated with long term domiciliary oxygen”, Respir. Med., 92(5), 287-291, 1997) and others that patients on oxygen therapy spend an average of 25% of time with an oxygen saturation (SpO₂) below the recommended limit of 90%.

The use of the system of the present invention is capable of providing an improved oxygen therapy regime, where the oxygen provided is determined by the patient's actual needs, reducing the amount of time spent in a hypoxic condition.

The device and system of the present invention can be utilized to improve home oxygen therapy (supplementary or long term), diagnosis and prescription in the doctor's surgery or elsewhere and ambulatory administration of oxygen, including in an emergency situation.

A particular benefit of providing this system for oxygen therapy in an emergency environment is that it is often uncertain the appropriate oxygen dose required by the patient, since there is insufficient time to carry out a detailed assessment. Since it is known that over-provision of oxygen can result in detrimental effects, it is appropriate to use a system that not only provides supplemental oxygen according to requirements, but prevents or minimizes over-oxygenation.

In providing oxygen therapy to a patient, the target may typically be set at a value above 90% (corresponding to a patients SpO₂ as measured by pulse oximetry), for example within the range of form 90-95% (i.e. a single value within the range or the whole range as the target range), such as about 93%. The saturation sub-component may be set to limit the flow of oxygen to pre-determined limits, such as from 0 to 5 litres per minute or 1 to 4 litres per minute, for example.

In an embodiment for the delivery of oxygen to a patient, the filter can be set to remove signals having a higher frequency than, for example, the lag between administration and change in blood-oxygen saturation (e.g. up to 50 seconds, preferably up to 30 seconds) or to prevent changes in oxygen delivery being made more frequently than the patient breathes, (e.g. fewer than 12 times per minute).

A further embodiment of the invention provides a system according to the invention and preferably a controller according to the inventions with a means for remote communication, for communicating data handled by the system or controller to another location. This means for remote communication may be a modem, especially a wireless modem, an infra-red device or Bluetooth® device or other wired or preferably wireless electronic communication means. According to this embodiment, a patient's drug regime may be monitored remotely by a doctor, health professional or computer/database to ensure that the medication is effective, to re-prescribe or to alert to unusual circumstances. Optionally, the controller or other component of a system includes the capability of receiving remote signals, via the same or different remote communication device, which will enable a health professional to alter the settings of the drug delivery system, device or controller to improve the therapy or according to the changing circumstances of the patient.

The system, device and controller of the present invention find application in any utility where there is problem with response time and stability in systems with long feedback time delays or time lag, saturation limits (safety or physical limits) and/or signal noise.

The invention will now be described, without limitation as to the scope of the invention, with reference to the attached figures and the following examples.

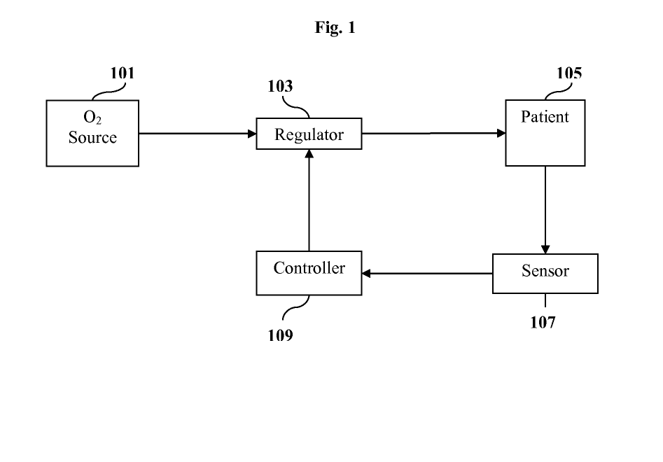

With reference to FIG. 1, a delivery system illustrated has an oxygen source 101 in fluid communication with a regulator 103, itself in fluid communication with a patient 105 to enable oxygen to be delivered in a regulated manner to the patient. The regulator 103, which regulates the flow of oxygen to the patient 105 in a controllably variable manner, is capable of receiving a control signal from the controller 109 which generates the control signal in response to a signal (a feedback signal), corresponding to a biochemical, biological or physiological property of the patient, received by the controller 109 from a sensor 107 configured to measure such a property of the patient. By utilizing a controller as described herein, the feedback system illustrated in FIG. 1 is capable of providing improved drug (for example oxygen) dosage according to the patients variable need as indicated by a measure of a patient biochemical, biological or physiological property.

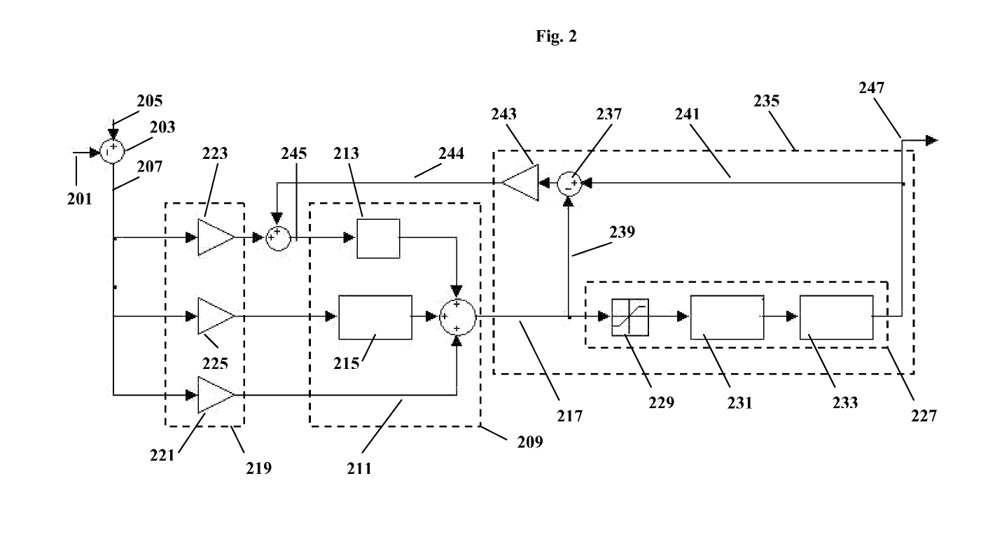

With reference to the controller in FIG. 2, the controller automatically controls the delivery of a drug or the flow of gas, especially oxygen, to a patient through a regulator, such as a continually variable solenoid valve. A signal input 205 receives an input signal from a sensor, such as a pulse oximeter (not shown). A comparator 203 generates an error signal 207 from the difference between the input signal and a target (201), which can be set via a user interface (not shown) or pre-programmed or hard-wired into the controller. An active component 209 generates an active output 217 after tuning of the error signal 207 by tuner 219. The active component 209 comprises a proportional sub-component 211, which provides a proportional response to the error signal, an integral sub-component 213 which provides an integral response to the error signal and a differential sub-component 215 to provide a differential response to the error. The responses from each of the sub-components of the active component 209 are combined to form the active output signal 217. The tuner comprises a proportional gain 221 for amplifying the error signal for the proportional response, an integral gain 223 for amplifying the error signal 207 to generate a pre-integration signal 245 for treatment by the integral sub-component 213, and a differential gain 225 for amplifying the error signal for the differential response. The precise tuning for any particular system depends upon a number of factors including the sensitivity of the sensor and the subject or patient, the characteristics of the drug being administered and the dynamics of the system, including the features of the manipulation component 227. The skilled person in the art should be capable of tuning the gains for any particular system to optimize the performance of the device.

Manipulation component 227 manipulates the active output 217 to generate an improved response transmitted as a final output signal via signal output 247. The manipulation component comprises a saturation (or limit) sub-component 229, which sets limits on the output signal. These limits might be physical (e.g. the physical limits of the valve) or safety (e.g. safe limits for administration of a drug to a patient). For oxygen therapy, the saturation (or limit) sub-component may have limits, for example, corresponding to an oxygen delivery rate of from 0 to 5 litres per minute.

The manipulation component 227 further comprises a filter sub-component 231 and a phase compensator 233. The filter sub-component 231 is preferably a low-pass filter for removing high frequency signals, such as a Butterworth first order filter. The settings on the filter 231 may be designed to disregard signals of a frequency that suggests they are not a consequence of the activity (the drug delivery) being controlled given the inherent lag in a sensor measuring the change in parameters or in the physiological response influenced by the controlled activity.

The phase compensator 233 compensates for phase lag caused by the manipulation components and especially by the filter 231 and is advantageously situated “downstream” from the filter 231.

One or more, and in this case all three, of the sub-components of the manipulation component should be within the anti-wind up component 235 which has a disparity component 237 which takes the difference from the post-manipulation signal 241 and the pre-manipulation signal 239 and feeds it via an appropriately tuned amplifier 243 to the pre-integration signal 245 to dissipate wind up in the integrator component.

By applying the controller described in FIG. 2, an improved rapid and accurate dosage of drug, e.g. oxygen, may be delivered to a patient in spite of the inherent lag times associated with the effect of a dose on physiological parameters, thereby enabling improved treatment and improved health in a patient.

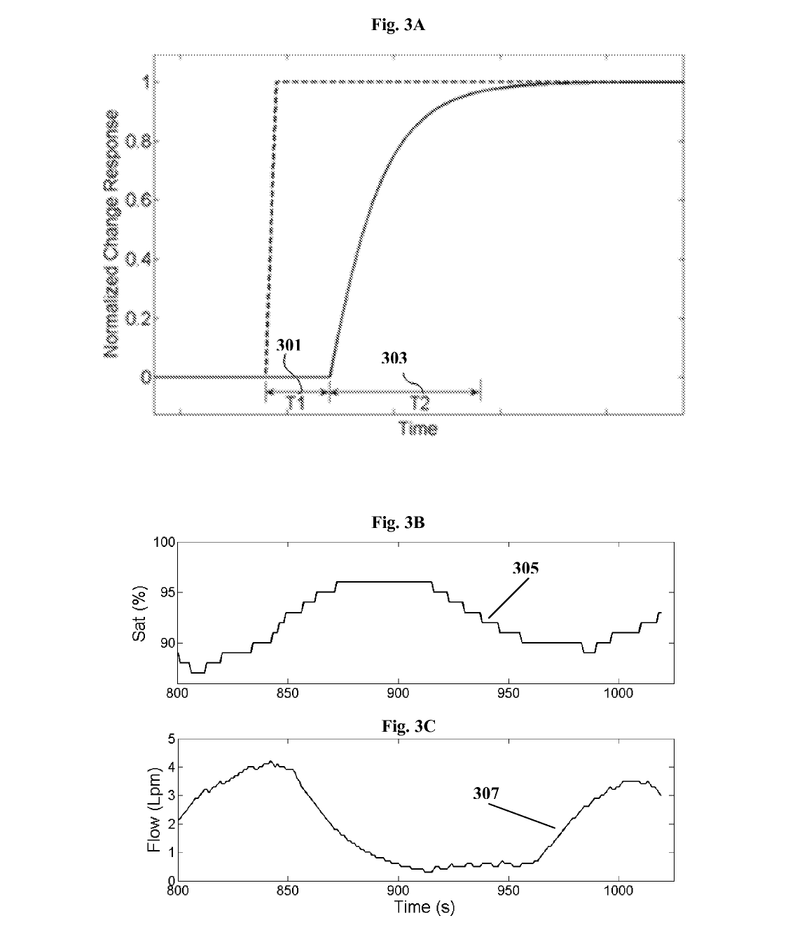

FIG. 3A is an illustration of a response by a measurable parameter (solid line) to a change to an activity influencing that parameter (dashed line) in a system characterized with a delayed time response.

FIG. 3B illustrates a variation in a patient's arterial blood oxygen saturation with time when receiving oxygen via a feedback control system not able to cope with the time delayed response;

FIG. 3C illustrates a variation in oxygen flow via a feedback control system not able to cope with the time delayed response, which is delivering oxygen to the patient referred to under FIG. 3B.

### EXAMPLES

### Example 1

The preclinical feasibility of the feedback controller was first demonstrated via a computer model using a closed-loop control algorithm to maintain a predetermined target. The controller was evaluated using a model to replicate the patient oxygen saturation response. This preclinical research was presented at the 2005 European Medical and Biological Engineering Conference (EMBEC).

The model replicated the patient oxygen saturation response described by the oxyhaemoglobin dissociation curve, which also incorporated a second order transfer function with fixed dead and lag times. Disturbances were input into the patient model to represent patient fluctuations in oxygen saturation. Depending upon the input arterial blood-oxygen saturation, the controller automatically regulates the oxygen flow between the gas source and the patient. Preliminary patient data was obtained from three Chronic Obstructive Pulmonary Disease (COPD) subjects during overnight monitoring with pulse oximetry at Royal Brompton Hospital, London. These recordings were then used as the input fluctuations to the controller simulation. The results indicate the potential to implement automated flow-rate control and correct fluctuations in oxygen saturation.

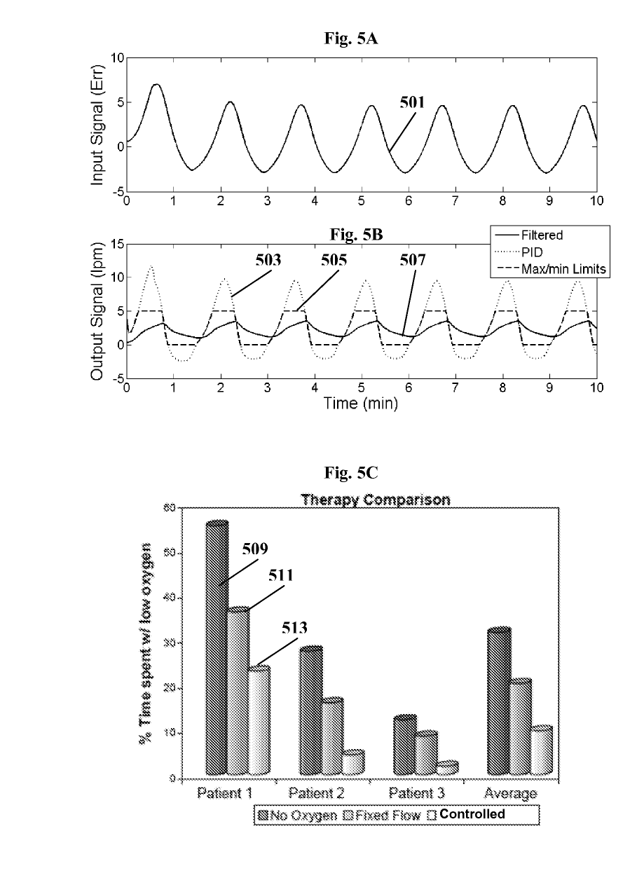

Parametric data computed from the patient records and simulation results are presented in FIG. 5C. The Controlled group (513) represents the results of automatically regulating the O₂ flow via the feedback controller. A second simulation was included to represent Fixed-Flow oxygen therapy (511). The oxygen flow-rate was normalized with respect to the SDOT group to yield equivalent oxygen consumptions. These simulation results indicate that the control system of the present invention is capable of substantially reducing, by 63% on average, the percentage of time a patient spends with a low blood oxygen level as compared with traditional oxygen therapy.

### Example 2

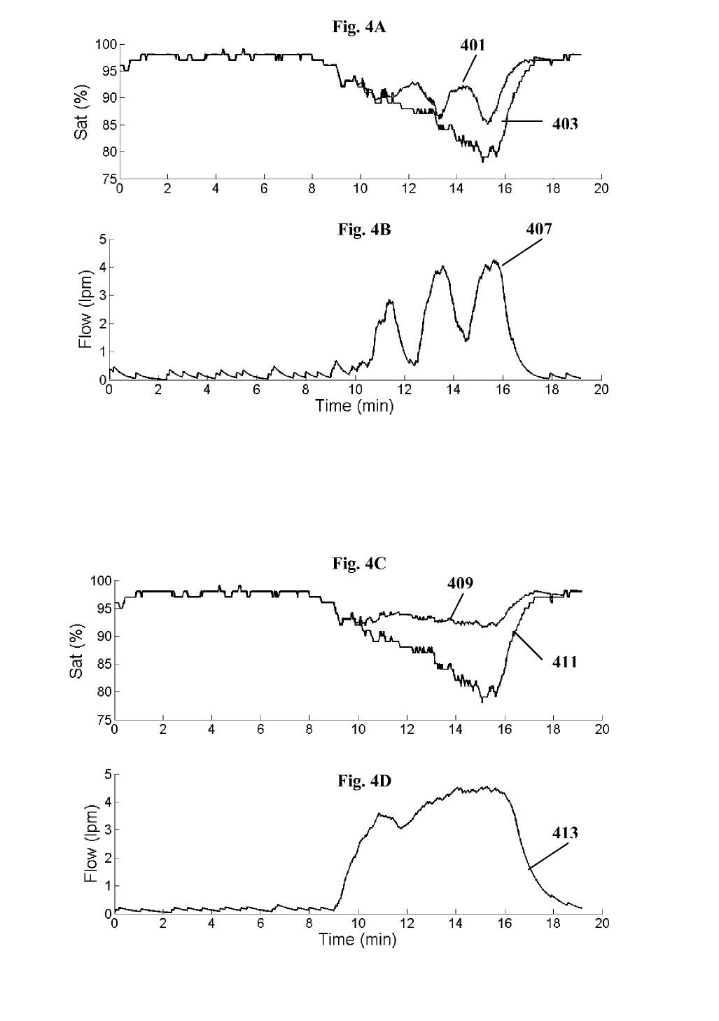

The results presented below regarding FIGS. 4A-D are derived from pulse oximetry monitoring during a standardised incremental shuttle walk exercise test. Shuttle walks are routinely used as simple clinical assessments of a patient's exercise ability. A 10 m shuttle course is outlined along a hospital corridor. Patients are instructed to walk along the course, turning at either end until too tired or breathless to continue. Pulse oximetry data was recorded continuously throughout the study period. The patient recorded oxygen saturation (403) was used as the input for a controller simulation to a linear control algorithm as described in the prior art without sufficient consideration for the time delay in the feedback response. In FIG. 4B, the oscillating behaviour of the linear controller is evident in the oxygen flow rate (407). The undesirable flow control oscillation can also be seen in the resulting saturation (401) of FIG. 4A. When the linear controller cannot maintain the predetermined target, the result is an unstable alternating flow between too much and too little oxygen delivery. Such unstable behaviour is characteristic of long feedback delays in linear control systems. Slow response and/or long feedback delays are a common issue particularly in various forms of non-invasive monitoring such as pulse oximetry or transcutaneous gas monitors.

The closed-loop drug delivery device described herein is advantageous in that it has a rapid and stable feedback response despite long feedback delays (FIG. 4C,D). Using the same patient input recording (411), the inventive controller flow output (413) dose not exhibit any potentially detrimental oscillatory behaviour. The inventive controller saturation result (409) shown in FIG. 4C predict a substantial improvement in the patient saturation, remaining close to the predetermined target.

The invention has been described with reference to a preferred embodiment. However, it will be appreciated that variations and modifications can be effected by a person of ordinary skill in the art without departing from the scope of the invention.

1. A drug delivery device for regulating delivery of a drug to a patient, the device comprising:
a regulator for controllably varying the rate of delivery or dose of a drug passing to the patient;
at least one sensor for measuring a biochemical, biological or physiological property of the patient, said property being associated with the drug to be delivered or with a condition to be treated by the drug to be delivered, and generating a signal corresponding to said measured property;
a controller, in communication with the at least one sensor and the regulator, configured to control the rate of delivery or dose of the drug provided by the regulator in response to one or more measured biochemical, biological or physiological properties provided by the at least one sensor with respect to a target,
the controller comprising
a comparator having a signal input configured to receive an input signal from at least one sensor and being capable of generating an error signal corresponding to the disparity between a target value and an input signal;
an active control component for generating an active output signal in response to the error signal; and
a manipulation component for manipulating the active output signal and generating a post-manipulation signal for communication to the regulator as a final output signal,
characterized in that:
the controller further comprises an anti-wind up component for reducing or inhibiting the effect of signal wind up in the active control component; and/or
the manipulation component comprises a filter sub-component for conditioning the active output signal,
whereby the final output signal provides a controlled rate of delivery or dosage administered to a patient in response to the measured property.

2. A device as claimed in claim 1, wherein the biochemical, biological or physiological property being measured by the at least one sensor is time-lagged or time-delayed with respect to the delivery or dose of drug with which it is associated.

3. A device as claimed in claim 1, wherein the anti-wind up component comprises a signal disparity component, which generates an anti-wind up signal from the difference between a pre-manipulation signal and a post-manipulation signal, and, optionally, a signal gain for generating an amplified anti-wind up signal, which may be fed into the active control component.

4. A device as claimed in claim 1, wherein the active control component of the controller comprises a proportionally responsive sub-component for providing a proportional response to the error signal and an integrally responsive sub-component for providing an integral response to the error signal, which active control component generates an active output signal corresponding a combination of the responses provided by the sub-components of the active component and wherein the controller further comprises an error signal tuner, which comprises a proportional gain to generate an amplified proportional signal, and an integral gain to generate a pre-integration signal.

5. A device as claimed in claim 3, wherein the active control component further comprises a differentially responsive sub-component for providing a differential response to the error signal and wherein the error signal tuner further comprises a differential gain to generate an amplified pre-differentiation signal.

6. A device as claimed in claim 1, wherein the controller comprises an anti-wind up component as defined in claim 1.

7. A device as claimed in claim 1, wherein the manipulation component comprises one or more of
a saturation (or limit) sub-component for adjusting the active output signal or pre-saturation signal to generate a post-saturation signal falling within predetermined limits;
a filter sub-component for conditioning the active output signal or a post-manipulation signal; and
a phase compensator, for compensating for signal phase lag generated within the system,
one or more of which sub-components are located within the anti-wind up component, whereby the disparity component generates the anti-wind up signal from the difference between a signal before the one or more sub-components and a signal after the one or more sub-components.

8. A device as claimed in claim 7, wherein the manipulation component comprises of the saturation sub-component and the filter sub-component, which are both located within the anti-wind up component.

9. A device as claimed in claim 7, wherein the filter sub-component is a low-pass filter

10. A device as claimed claim 1, wherein the regulator comprises a valve.

11. A device as claimed in claim 10, wherein the valve is a proportional solenoid valve.

12. A device as claimed in claim 1, wherein the drug is a medical gas.

13. A device as claimed in claim 12, wherein the drug is oxygen and the device is for the regulated administration of supplementary oxygen to a patient.

14. A device as claimed in claim 13, wherein at least one sensor is a pulse oximeter, which generates a signal corresponding to the level of oxygen saturation of a patient's arterial blood.

15. A device as claimed in claim 1, which comprises a user interface for the setting of a target.

16. A delivery system for regulated administration of a drug to a patient, the system comprising:
a source or reservoir of a drug;
a drug delivery means for the passage of a drug from the source or reservoir to a patient; and
a drug delivery device as defined in claim 1, which is configured such that the regulator is for controllably varying the rate of delivery or dose of drug passing to the patient via the drug delivery means from the source or reservoir.

17. A system as claimed in claim 16, wherein the biochemical, biological or physiological property being measured by the at least one sensor is time-lagged or time delayed with respect to the delivery of drug with which it is associated.

18. A system as claimed in claim 16, wherein the drug is a medical gas.

19. A system as claimed in claim 18, wherein the drug is oxygen and the system is for the regulated administration of supplementary oxygen to a patient.

20. A system as claimed in claim 19, wherein the oxygen source is an oxygen cylinder, dewar or oxygen concentrator.

21. A system as claimed in claim 19, wherein at least one sensor is a pulse oximeter, which generates a signal corresponding to the level of oxygen saturation of the patient's blood.

22.-29. (canceled)

30. A method of administering a drug to a patient comprising the steps of:
providing a source or reservoir of a drug;
providing and fitting to a patient a delivery means for passage of the drug from the reservoir or source to the patient;
providing a device as defined in claim 1 configured to control the passage of the drug form the reservoir or source to the patient via the drug delivery means and fitting the at least one sensor of the device to the patient and configuring said sensor to measure a biochemical, biological or physiological parameter to be influenced by the administration of the drug;
administering a dose or delivery rate of the drug to the patient as regulated by the device; and causing the controller of the device to control administration of drug to the patient in response to signals generated by the sensor,
whereby the rate of delivery or dosage administered to the patient is such as to provide a controlled influence on the measured biochemical, biological or physiological property as desired.

31. A method as claimed in claim 30, wherein the drug is oxygen, the sensor is a pulse oximeter and the device has a regulator, which is a valve.

32. A method as claimed in claim 30, wherein the delivery means comprises tubing connected to an oxygen face mask or nasal cannula.

31-36. (canceled)
